(19) United States
(12) Patent Application Publication
(10) Pub. No.: US 2015/0245767 A1
(43) Pub. Date: Sep. 3, 2015

(54) DUAL IRIS AND COLOR CAMERA IN A MOBILE COMPUTING DEVICE

(71) Applicant: LRS Identity, Inc., San Mateo, CA (US)

(72) Inventors: Malcolm J. Northcott, Felton, CA (US); Keith W. Hartman, Redwood City, CA (US); Joseph Justin Pritikin, Mountain View, CA (US)

(21) Appl. No.: 14/635,771

(22) Filed: Mar. 2, 2015

Related U.S. Application Data
(60) Provisional application No. 61/946,340, filed on Feb. 28, 2014.
(60) Provisional application No. 61/973,116, filed on Mar. 31, 2014.

(51) Int. Cl. A61B 3/12; A61B 3/00

(52) U.S. Cl. CPC A61B 3/1216; A61B 3/0008

(57) ABSTRACT

A dual purpose iris and color camera system is described provides good iris and color image capture in either IR or visible bands depending upon which type of image is being captured at that moment. For iris imaging the iris camera is capable of imaging in the 700 to 900 nm wavelength range where the iris structure becomes visible. The iris camera is able to perform iris imaging outside with full sunlight. The iris camera requires only a low level of cooperation from the user, in that they must be within a range of distances away from the iris camera, must hold relatively still for a short period of time, and must face towards the camera. The iris capture process is fully automated once activated.

### CROSS REFERENCE TO RELATED APPLICATIONS

This application claims the benefit of U.S. Provisional Application No. 61/946,340, filed Feb. 28, 2014, which is incorporated by reference herein in its entirety. This application also claims the benefit of U.S. Provisional Application No. 61/973,116, filed Mar. 31, 2014, which is also incorporated by reference herein in its entirety.

### BACKGROUND

Iris imaging systems capture images of the human iris for a variety of purposes, examples of which include biometric (human subject) identification as well medical imaging. As a significant proportion of the human population has brown eyes, iris imaging systems generally must be able to image irises for subjects with brown eyes. Melanin pigment in brown eyes becomes transparent at 850 nm, which is just outside the visible range in the near-infrared (IR) spectrum. Consequently, iris imaging systems generally function by imaging light at and around these near IR wavelengths.

Beyond this and other basic requirements, iris imaging systems vary significantly depending upon the demands of the system. Systems assuming a cooperative human subject who is willing to be positioned very close to the imaging apparatus are easier to design. On the other hand, imaging systems designed for uncooperative subjects located a non-trivial distance away (e.g., on the order of tens of centimeters to upwards of a few meters) are generally more complicated, and must address focus and ambient light issues. For example, to construct an iris imaging system that works successfully for outside imaging, the system must include a mechanism for eliminating specular reflections from the outside light sources that would otherwise interfere with iris imaging. One way to accomplish this goal features a light filter that becomes transparent at or near the 850 nm wavelength.

This solution introduces a problem, however, for constructing an iris imaging system that can also act as a normal camera (herein referred to as a color camera), such as might be integrated into a modern smart phone. The typical color camera in a smart phone uses a complementary metal-oxide semiconductor (CMOS) image sensor (detector) that is overlaid by a Bayer filter for separating capturing red, blue, and green (RGB) light into different pixels/subpixels. All three colors of the Bayer filter typically become transparent at or around the 850 nm wavelength at which iris imaging is performed. FIG. 1 is an example illustration of the transparency of color filters in a typical prior art Bayer filter.

Due to this property, most color cameras include a separate IR blocking filter that is used to prevent IR illumination reaching the detector. Without this blocker, in situations where IR radiation is present (e.g., outdoors) color images will appear to have low color saturation and a pinkish tint. The pinkish tint is due to the red filter being more transparent in the IR. The IR filter may be omitted in cameras where sensitivity is more important than color rendering. Examples includes surveillance cameras and automotive rear view cameras. However, this represents a tradeoff rather than a solution to the problem.

### BRIEF DESCRIPTION OF THE DRAWINGS

FIG. 1 is an example illustration of the transparency of color filters in a typical prior art Bayer filter.

FIG. 2 illustrates an imaging system for capturing iris images, according to one embodiment.

FIG. 3 is a plot of filter transparency as a function of wavelength for a notch IR filter for use with the imaging system, according to one embodiment.

FIG. 4 illustrates the spectral transmittance of an example photochromatic filter, according to one embodiment.

FIG. 5 illustrates the illumination spectrum of an LED near IR illuminator, according to one embodiment.

FIG. 6A illustrates the throughput a near IR illuminator/notch IR filter combination, according to one embodiment.

FIG. 6B illustrates the relative filter throughput of the near IR illuminator/notch IR filter combination as a function of filter FWHM, according to one embodiment.

FIGS. 7A and 7B illustrate two different views of the approximate charge collection regions from a FOVEON X3 stacked set pixel detector, according to one embodiment.

FIG. 8 illustrates an example modified Bayer filter to include color and near IR filters, according to one embodiment.

FIG. 9 illustrates a process for capturing iris images using WOI controls, according to one embodiment.

FIG. 10 plots the SNR for red, green, and blue Bayer filters of the imaging system as a function of exposure time, according to one embodiment.

FIGS. 11A and 11B illustrates example scaled black body spectral distribution plotting Power (Watts per unit volume) and Photons (Count of photon flux per unit volume) as a function of wavelength for use in an SNR calculation of the imaging system, according to one embodiment.

### DETAILED DESCRIPTION

### 1. System Overview

1.1. General Overview

A dual purpose iris and color camera system is described provides good iris and color image capture in either IR or visible bands depending upon which type of image is being captured at that moment. For iris imaging the iris camera is capable of imaging in the 700 to 900 nm wavelength range where the iris structure becomes visible. The iris camera is able to perform iris imaging outside with full sunlight. The iris camera requires only a low level of cooperation from the user, in that they must be within a range of distances away from the iris camera, must hold relatively still for a short period of time, and must face towards the camera. The iris capture process is fully automated once activated.

1.2. Imaging System

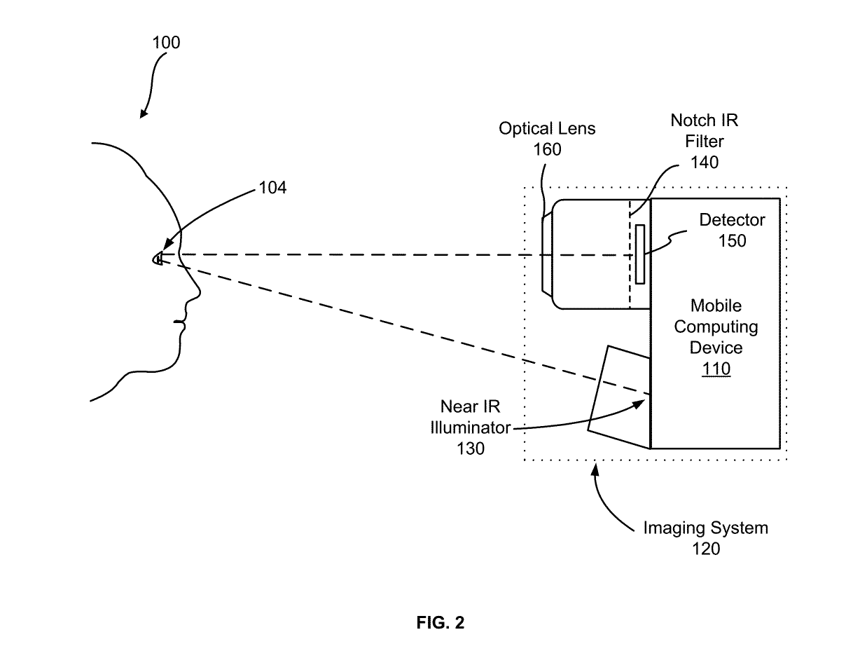

FIG. 2 illustrates an imaging system 120 for capturing iris images, according to one embodiment. The system is configured to capture at least a pair of images of a subject's 100 eyes 104 including a background image without IR illumination and an IR image under IR illumination, and subtract the one or more pairs of images to generate an iris image. The imaging system 120 includes a mobile computing device 110 such as a smart phone, a near IR illuminator 130, an optical lens 160, a notch IR filter 140, and an imaging sensor (detector). Although only one of each component is shown, in practice more than one of each component may be present.

The optical lens 160 transmits light reflected from the subject's 100 eyes 104 towards the detector 150, and can be controlled, for example by the mobile computing device 110, to change its optical power (e.g., the inverse of the focal length of the imaging system 120, often quantified in diopters) to capture images at multiple different positions. In one implementation, the optical lens 160 is a liquid lens that can vary its focal length in nearly any increment by application of an electric field to the elements of the liquid lens. One advantage of the liquid lens 110 is its extremely fast focus-adjustment response time, approximately 20 milliseconds, compared to lenses using mechanical means to adjust the focus. This is particularly advantageous for capturing focused images of irises quickly for any subject, particularly for uncooperative subjects that may be resisting identification. Another optical element that can be focused as quickly and used in place of a liquid lens is a deformable mirror. Furthermore, the optical lens 160 may include, or be in optical communication with, a multi-element lens (not shown) used for zooming the field of view of the imaging system 110 to the eyes 104. In one example, the field of view is a 256 pixel×256 pixel field of view, but other examples can have larger or smaller fields of view.

The optical lens 160 partially or completely focuses received images onto the detector 150. The detector 150 is substantially disposed in the focal plane of the optical lens 160 and is substantially perpendicular to the optical axis of the imaging system 120, thereby allowing an image of the iris to impinge upon the detector 150.

The notch IR filter 140, detector 150, and components of the mobile computing device 110 that allow for capture and processing of iris images are described further below. Particularly, the mobile computing device 110 includes a computer processor, computer storage device (e.g., a hard drive or solid state drive (SSD)), a working memory (e.g., RAM), computer program cod e (e.g., software) for performing the operations described herein, a visual display, a user input device such as a touchpad, and may also include a separate color camera using a different detector than detector 150. These components allow for user input to control the image capture process, and also allow for the automation of the entirety of the image capture process for triggering to storage of an iris image on the mobile computing device 110. The mobile computing device may also include a wireless transceiver (e.g., an 802.11 or LTE processor) for communicating iris images to an external computer server.

The various components described above can be attached to (or held together) by a frame (not shown). This may be the housing of the mobile computing device 110, such that all components of the imaging system 120 are contained within the housing of the mobile computing device 110. Alternatively, components of the imaging system 120 other than the mobile computing device 110 may be removably attached to the mobile computing device 110.

### 2. Notch IR Filter

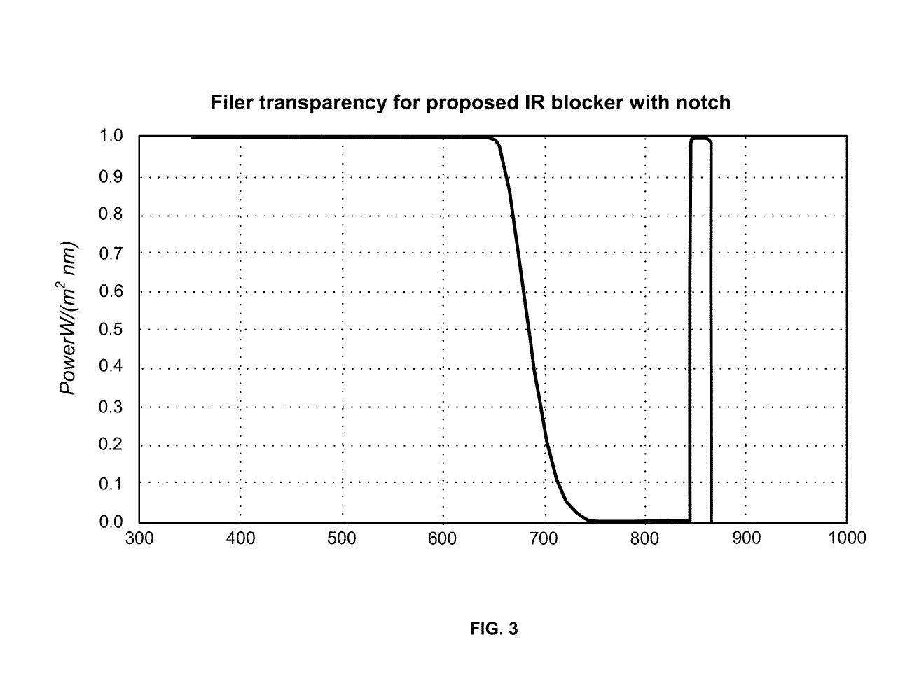

FIG. 3 is a plot of filter transparency as a function of wavelength for a notch IR filter for use with the imaging system, according to one embodiment. The dual imaging system is responsive enough to illumination in the near IR to capture iris images with good signal to noise ratio (SNR). However, the detector also contains mechanisms to address color distortion for portrait images. In one implementation, these two seemingly antagonistic requirements can be met by exploiting the narrow bandwidth of the IR illumination sources that are needed to illuminate the iris for capturing iris images.

In this implementation, an IR blocking filter 140 is placed in the optical path between the subject and the detector, where the IR blocking filter has a small transmission notch centered at the wavelength of the iris imaging system's IR illuminator. In one embodiment, this notch has a full width half maximum (FWHM) at 20 nm wide centered either at 780 or 850 nm, or centered within 20 nm of either 780 or 850 nm. However, the notch may be wider or narrower and centered on another wavelength, depending upon the implementation. For example, if the near IR illuminator is wider band (e.g., an LED), a wider notch (e.g., FWHM of 20 nm) may be used to accommodate the expected return light reflected off of the iris. Similarly, if the near IR illuminator is narrower band (e.g., a laser), a narrower notch (e.g., FWHM of 10 nm or less) may be used. The notch IR filter (or simply notch filter) allows a significant IR iris signal to be recorded without seriously distorting the color balance of color images in an outside environment.

The notch filter 140 may also be constructed to include two or more transmission notches, each centered to transmit a different wavelength. For example, a first transmission notch could be centered at 850 nm and another transmission notch could be centered at 780 nm. In such an implementation, the imaging system 120 would include multiple illuminators, each having a center wavelength chosen to match a center wavelength of one of the transmission notches. The FWHM of each transmission notch would be chosen to be appropriate for the associated illuminator, (e.g., FWHM for transmission notch associated with an LED illuminator would be wider than the FWHM for the transmission notch associated with a laser illuminator).

In one embodiment, the imaging system further reduces background solar illumination by either configuring the notch IR filter to block telluric absorption lines, or by including a second filter that blocks telluric absorption lines.

The notch IR filter may be a switchable filter that allows the imaging system to control whether or not the filter affects captured images. In a simple embodiment, this may be a mechanical actuation mechanism to move the filter into and out of the optical path between the detector and the subject. Alternatively, the filter may be activated or deactivated using an electrical switch without being physically moved. With a switchable filter, the combination of near IR bandwidth, exposure time, and near IR illuminator brightness can be tuned to reject environmental reflections when desired.

2.1. Notch Filter Color Distortion Using Solar Illumination.

Like any other filter a notch filter can distort the color balance of portrait images captured in that mode. However, a notch filter generates relatively little distortion compared to other kinds of filters. In an example circumstance where the imaging system, including a Bayer color filter and a notch IR filter captures an iris image in daylight, the amount of distortion can be determined based on the rate detected photoelectrons impinging on each of the color filters, according to:

N=Alₚ²πrₗ²/2πl_{z}²∫n(λ)Q(λ)f_{c}(λ)fᵢᵣ(λ)dλ (1)

where N is the number of detected photoelectrons per second, A is the Albedo of the object being imaged, lₚ is the side length of a pixel (projected on the object), r₁ is the radius of the imaging lens aperture, l_{z} is the object distance from the lens aperture, n(λ) is the black body spectrum expressed as number of photons per unit wavelength per second, Q(λ) is the quantum efficiency of the detector as a function of wavelength, includes any losses in the optics, f_{c}(λ) is the throughput of the color filter, and fᵢᵣ(λ) is the throughput of the IR filter. where all parameters are the same as in equation (5) above except n(λ) is the black body spectrum expressed as number of photons per unit wavelength per second. The computed throughputs of the Bayer are shown in Table 1, assuming an albedo of 0.1.

TABLE 1

| | N_{R} through | N_{G} through | N_{B} through |
|---|---|---|---|
| Filter | red pixel | green pixel | blue pixel |
| Configuration | filter | filter | filter |
| With IR blocker | 526 | 553 | 224 |
| With 20 nm wide | 563 = | 584 = | 257 = |
| IR notch | 526 + 37 | 553 + 31 | 224 + 33 |
| With no filter | 1237 = | 769 = | 450 = |
| | 526 + 711 | 553 + 216 | 224 + 226 |

In the absence of an IR filter, the red pixel and blue signal pixel count rates are increased by a factor of 2 by solar illumination. This large signal gives the image a red-purple cast and significantly reduces color saturation. Known automated white balance algorithms are not able to cope with this large additional signal.

By contrast using a 20 nm wide notch filter where the notch is located at or near 850 nm increases the red pixel signal by about 7%, and the blue pixel signal by about 15% relative to using a total IR blocker filter. These relatively modest increases in signal will not disturb the color balance or saturation significantly, and can be corrected by normal white balance algorithms.

Different object albedos will move the signal level, but will generally not change the ratio of signals with respect to the various filter options as the albedo is usually wavelength independent. For some objects this wavelength independence may not hold true, and as a result some subtle color changes may still be observed in portrait images where the notch filter is present. However, even Bayer color filters do not exactly match the response curves of the eye's color receptors, and thus some small amount of color distortion is acceptable as inevitable.

2.2. Photochromic Filters

Photochromic materials use the UV light to reversibly change the structure of a dye to turn it from clear to opaque. In the absence of UV light, the dye will return to the clear state. A property of many photochromic dyes is that the absorption is fairly uniform at visible wavelengths, but much less pronounced in the near IR spectrum, such as at 850 nm.

Since environmental reflections are a potential source of SNR loss in iris imaging, a photochromic filter can be used to reduce the relative intensity of visible wavelengths compared to near IR wavelengths when capturing iris images outside. As the imaging system's detector is typically much more sensitive to visible light than to near IR light, introducing a photochromic filter between the detector and the subject effectively reduces the contrast of the environmental reflections on the cornea. An advantage of this approach is that it is completely passive, but does not impact the sensitivity of the detector in low light conditions. A disadvantage is that the photochromic reaction is not instantaneous, requiring anywhere from a few seconds to a few minutes for the filter to change state in response to different UV illumination levels.

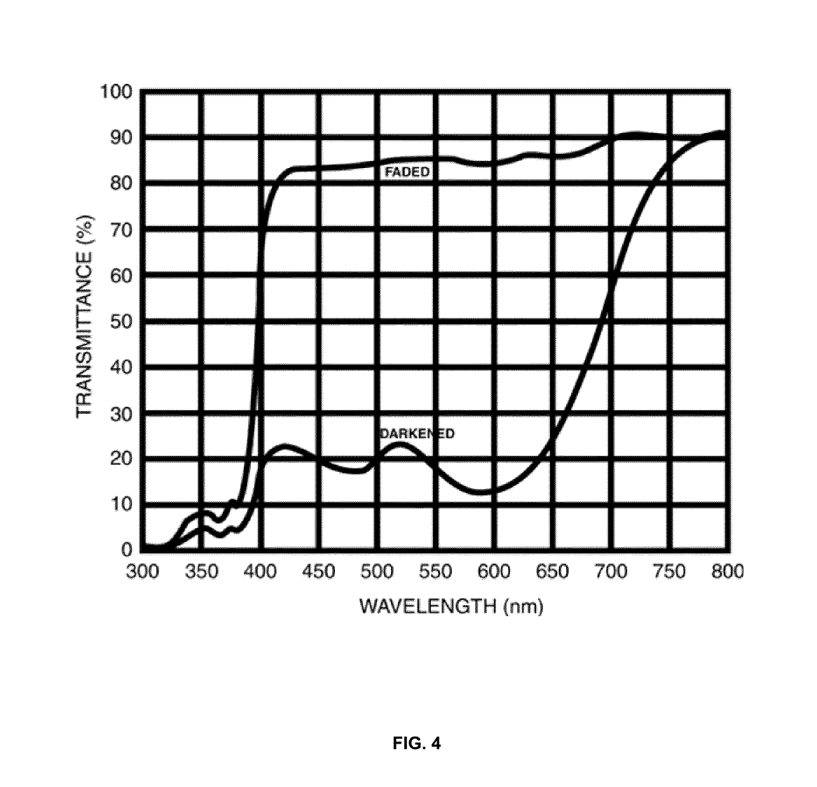

FIG. 4 illustrates the spectral transmittance of an example photochromatic filter, according to one embodiment. Iris image SNR can be improved by using a photo chromatic filter in conjunction with the notch filter. When activated the photochromatic filter is activated, the transmittance through the photochromatic filter for visible wavelengths is reduced by about a factor of 4, whereas transmittance at near IR wavelengths is virtually unchanged. This results in a 4× improvement in SNR versus not using a photochromatic filter.

The design of the imaging system may trade off of some or all of the SNR in order to instead reduce the total exposure time needed to make an iris image. For example, rather than holding exposure time constant to improve SNR by a factor of four, instead the exposure reducing the by a factor of approximately 4. However, the photochromatic filter also has the side effect of making near IR radiation more pronounced, thereby negatively affecting color balance. Thus, an imaging system including a photochromatic filter will take this into account, balancing between exposure time for an iris image, and color fidelity.

2.3. Near IR Illumination

To illuminate the human subject with near IR light for iris image capture, even in daylight, several different types of visible and near IR illuminators 130 may be used. The These include light emitting diodes (LEDs) (including organic light emitting diodes (OLEDs)), lasers including vertical-cavity surface-emitting laser (VCSEL) arrays, and other IR illuminators. The type of near IR illumination used affects the performance characteristics of the system. A few examples of near IR illuminators are described below.

Also as introduced above, there may be a combination of multiple illuminators used including near IR illuminators (e.g., at or around 850 nm) and illuminators near the boundary of the visible and NIR ranges (e.g., at or around 780 nm). For simplicity, these illuminators emitting light near the boundary of the visible and infrared range are also referred to as near IR illuminators, even though some of the wavelengths they emit may be in the visible spectrum.

The near IR illumination is strong enough to be clearly visible above the noise generated from the visible image, for example using short exposures with bright flashes, so that fluxes comparable to solar illumination can be generated for the short times over which exposure are taken.

Furthermore, because the imaging system 120 is configured for imaging irises, the near illuminator 130 can be configured to produce a dual-lobed irradiance or illumination distribution, wherein the lobes of the distribution are located approximately at the eyes 104 of a subject separated from the near IR illuminator by the standoff distance. The standoff distance is the distance separating the imaging system 120 and the subject 100. This configuration can use any combination of lateral or angled separation of the near IR illuminator, calculated using geometry principles, to produce the dual-lobed irradiance distribution at the standoff distance.

The near IR illuminator may also include its own filter for narrowing the wavelength of light that reaches the subject's eyes. This can allow for more efficient discrimination of extraneous background images from the iris image. For example, when used in cooperation with the notch IR filter, described above, ambient illumination can be suppressed, thereby emphasizing the corneal glints reflected from the eyes of the subject. The near IR illuminator may also include a lens (not shown) to further focus, defocus, or otherwise direct light from the near IR illuminator to the eyes 104 of the subject 100. The lens can be used to tailor the shape and/or intensity of the light distribution at the standoff distance or at the various focal points. One embodiment would be to use a four-element liquid lens to steer and focus the NIR illumination. The steering target would be the glint (the highly reflective image of the illumination source in the cornea). The standoff distance would be computed from, for example, a contrast-based focus metric. The illuminator intensity could be dynamically adjusted to provide a constant light intensity on the surface of the eye. Such a system would provide for a constant exposure for eye-safety and minimize power consumption.

2.3.1. LED Near IR Illuminator

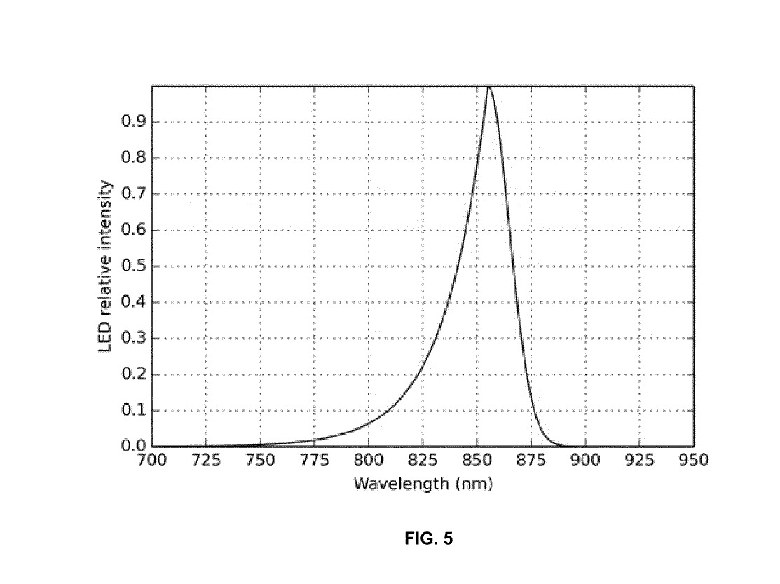

FIG. 5 illustrates the illumination spectrum of a near IR illuminator, according to one embodiment. A light emitting diode (LED) can provide near IR illumination for iris imaging. In one embodiment, the LED is an OSRAM LED. A typical LED illuminator has a band pass of about 40 nm, though relatively wide this is still about five times narrower than the band pass of typical Bayer color filters in the near IR wavelength range.

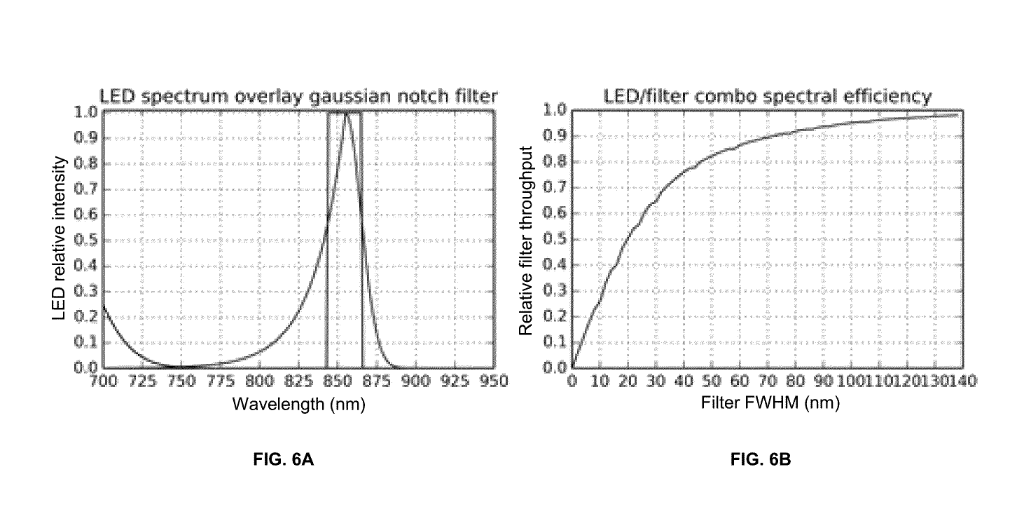

FIG. 6A illustrates the throughput an example near IR illuminator/notch IR filter combination, according to one embodiment. FIG. 6B illustrates the relative filter throughout of the near IR illuminator/notch IR filter combination as a function of filter FWHM, according to one embodiment. If the LED is paired with a notch IR filter over the detector, as introduced above, most background IR illumination is filtered out, thereby preventing background IR illumination from seriously impacting the effective illumination level produced by the near IR LED. In the example of FIG. 6A, the notch filter has a FWHM of 20 nm, providing roughly 50% throughput for the near IR LED illuminator's light. In other embodiments, different filter widths and different notch profiles could be chosen.

At the expense of requiring brighter near IR illumination, the band pass of the filter could be reduced further to a FWHM of less than 20 nm (e.g., 10 nm). Narrower filters progressively reduce the negative effects of near IR illumination on color balance, but work best with more near IR illumination available, such as if a laser (or laser array) illuminator is used, as described immediately below.

2.3.2. VcSEL Array Illuminator

The illuminator in the imaging system may be a laser, or an array of lasers such as a VCSEL array. A laser light source can be fabricated with a much narrower spectral bandwidth than a LED. Bandwidths less than 1 nm are easily achievable. This would allow for the use of a very narrow notch filter, and cut down IR contamination of visible images by more than a factor of 10 compared to an LED illuminator. The limit to achievable bandwidth narrowness is the practicality of building uniformly narrow band filters at reasonable price, the challenge of controlling wavelength drift with temperature, and controlling angular dependence of the filter bandwidth.

Laser illuminators also have the drawbacks of raising eye safety and spatial coherence concerns. When a laser is used as an illuminator, the system would have to comply with laser safety standards, such as ANSI Z136/IEC 60825 rather than lamp safety standards that apply to LED illuminators, such IEC 62471. While designing an eye safe class 1 laser near IR illuminator is feasible, regulations still require a laser sticker to be visible on the product. This can make a product including the imaging system to be undesirable from a consumer perspective.

A single laser used as a near IR illuminator would produce light with enough spatial coherence to cause speckle, which would effectively add noise at multiple spatial frequencies to the image. Increasing the exposure time would not reduce speckle noise significantly, and this might adversely affect the accuracy of the iris biometric. One possible solution to this problem would be to use an array of incoherent VCSEL or non-mode locked lasers as the near IR illuminator. The incoherent lasers in the VCSEL would significantly reduce the spatial coherence of the illumination and therefore reduce the speckle noise while maintaining the narrow spectral bandwidth.

### 3. Improving Detector SNR

As introduced above, one process for iris imaging involves taking two images close together in time, and then performing a subtraction to generate the iris image. Taking the images close together in time minimizes the amount of time for subject or camera motion to change, thus increasing the noise of the subtracted image.

3.1. Rolling Shutter Detectors

Many detectors used in modern mobile computing devices use a rolling shutter design to achieve exposure control. This means that the exposure of each successive line in the detector is delayed by one line readout time relative to the previous line. As a result, exposure and read times are staggered as a function of vertical position in the image. Typically a line readout time is of the order of 5 μsec.

One problem of using progressive read imagers to do image differencing is that the whole WOI (Window Of Interest) is read out sequentially. Furthermore when the near IR illuminator is turned on, it stays on for a time that is at least the sum of the WOI readout time plus the integration time. There are a number of problems arising from this extended illumination time. Firstly, the drive current of the near IR illuminator, and thus the peak illumination falls with pulse time. For instance if the frame readout time for a WOI is 2 milliseconds (ms), then the peak drive current for the diode is about 2.5 Amps (A). If the flash duration could be reduced to 200 μsec, then the peak drive could be increased to 5 A, increasing the contrast with the background.

Secondly, eye safety standard requirements dictate total power incident in the eye over a period of time, so the extended exposure time necessitates a shorter exposure in order to meet those standards. Short pulses can be considerably brighter than long pulses while maintaining eye safety. Although in general the near IR illumination used for iris imaging contemplated in this disclosure is well within the eye safety envelope, maintaining a large eye safety margin is good practice for a device that may be used on a regular basis. Thirdly, more energy will be used in a longer exposure pulse, which compromises the battery life of the mobile computing device.

Readout time for a progressive scan detector can be significantly reduced by providing several parallel readout channels. As many as all lines in the imager could have its own pair of readout amplifiers (one per color for the Bayer filter for each row). This would allow a 200 pixel line (plus 50 pixel over-scan) to be read out in about 2.5 μsec. Intermediate solutions could achieve smaller speedups by adding less readout amplifiers, with each readout amplifier handling either an interleaved sets of lines, or a dedicated block of lines. Interleaved lines would be more useful for speeding up WOI reads than dedicated blocks because it is more likely that all the added signal chains could be used independently of the size and position of the WOI. One disadvantage of adding additional readout amplifiers is that analog amplifiers and analog to digital conversion (ADC) components tend to be quite power hungry, potentially leading to significant heat and battery lifetime issues. One way to address this issue would be to enable signals to be routed to a single set of signal chains for regular use, powering on, and rerouting signals to the additional signal chains only when a rapid readout is required.

Additional signal chains could also be associated with on-chip binning controls, such that a single set is used when the detector is binned down to low resolution mode, and additional sets of signal chains come on line as resolution is increased. For instance a 640×480 video conferencing mode could use a set of 4 signal chains to run color video conferencing with a VGA image, with the chip internally binned in a 4×4 or 6×6, or another binning pattern. Assuming that the 640×480 is binned 4×4, then for iris imaging, captured near IR and background images could have a 1280×960 mode, utilizing 2×2 binning, and 16 independent signal chains. Finally, a non-binned mode of 2560×1920 with 64 independent signal chains could give full resolution.

3.2. Global Shutter Detectors

A global shutter detector may be used in place of a rolling shutter detector. In a global shutter detector, all pixels in the imager begin and end integration at the same time, however this feature requires at least 1 extra transistor to be added to each pixel, which is difficult to achieve with the very small pixels used in the detectors used in many mobile computing devices. Generally, this requires a slightly larger pixel pitch. However, if a detector supporting a global shutter feature is used, it would facilitate combined iris and portrait imaging. This is because it would allow for more accurate synchronization of near IR illumination and the image exposure, as the entire exposure could be captured at once. As a result, the near IR illuminator could be driven at higher power for less time. The higher power would in turn allow for a higher SNR in the subtracted iris image.

3.3. Charge Counter Pixel Design

Many modern detectors integrate photoelectron charge on a pixel for a set amount of time, and then transfers that charge to a readout amplifier to compute the signal. However, in one implementation the detector of the imaging system may be designed to include a very small full well by causing the output transistor gate on the pixel to have extremely low capacitance. This allows for a very high transcapacitance gain and therefore an extremely low read noise, in some cases less than the voltage signal of a single photoelectron. This type of detector does not include a traditional signal chain or an ADC.

If the detector has this structure, each pixel can be coupled to a comparator that is designed to switch after a given number of photoelectrons have been detected. When the integrated photo-current charge in a pixel has reached a predetermined level the comparator flips. When the comparator flips, it sends a pulse that increments a counter that maintains an increment total for each pixel, and also resets the pixel for a new integration cycle. In this way the dynamic range of detector is set only by the size of the counter. The benefit of this arrangement is that the image can be non-destructively read at any time simply by copying the content of the counter. In this way the detector can simulate a global shutter, thereby isolating the background image from the near IR image, while minimizing the duration of the flash.

A detector with this design allows for easy synchronization between effective image integration periods with periods where the near IR illuminator is turned on and off. A further advantage of this design is that it allows for extremely high dynamic range, limited only by the maximum counter rate. This would allow for imaging of the IR glint without loss of linearity, even though this glint would be highly saturated in a traditional detector. An unsaturated glint image allows for extremely precise image re-centering, and would provide an extremely high SNR point spread image which could be used to de-convolve the iris image to achieve even higher image quality than could be achieved with a traditional detector. The brightness of the glint can also be used to distinguish real eyes from photographs and from glass eyes.

3.4. Modified Double Correlated Sampling Circuitry

The detector may use a modified version of double correlated sampling to improve SNR. In traditional double correlated sampling, a pixel value is read after reset, then after the integration period is over, and the two values subtracted to estimate the pixel photocurrent. This process significantly reduces read noise by reducing 1/f noise that is characteristic of many detectors and readout circuits. The double correlation process may be carried out digitally or in analog depending on the architecture of the detector.

For iris imaging, double correlated sampling can be modified by reading the pixel after reset, then once again after an integration time during which the pixel is not illuminated by the near IR illuminator, then once more after the near IR illuminator has been flashed on. Carrying out the operations in this order without an intervening pixel reset will reduce the noise of the difference image.

3.5. Gain Setting with Small Form Factor Detectors

Newer generations of high resolution small format cameras have extremely small pixels. This results in very small capacitance for the pixel and therefore a very small full well, typically with a full well of the order of 20,000 photoelectrons or smaller, after which the detector signal becomes saturated. The most recent detectors also have very small read noise, typically of the order of 10 electrons RMS and often much lower. With a full well of 20,000 the maximum SNR obtainable on a pixel, due to photon noise is of the order of \sqrt{(20,000)} or about 140. Also many modern detectors have 12 bit converters on the output, which means each bit of gray scale corresponds to about 5 photoelectrons. For detectors such as this, if the gain of the system is arranged such that the maximum digital signal corresponds to the maximum full well, the digitization noise would be less than the read noise and photon noise for all signal levels. Under these circumstances there is no information benefit in adjusting the gain of the detector away from this optimal value. Furthermore, for all situations except the darkest images, the pixels are dominated by photon noise, and there is no significant penalty for spreading an exposure over multiple images.

3.6. Two or More Detectors with Dichroic Filters/Mirrors

Some detectors include three separate detector chips to independently sense red, green, and blue wavebands. Typically light is divided into different bands using a series of dichroic beam splitters, these may be built into a set of prisms to maintain stability of alignment. The imaging system could use such a structure to capture images for iris imaging, where a standard CMOS color detector chip shares a single lens with an IR detector chip. A dichroic beam splitter is used to direct the visible and IR wavebands to the color and IR detector chips, respectively.

3.7. Deep Depletion Detectors

Silicon detectors are built from PN semiconductor junctions. Electron-hole pairs are generated when a photon is absorbed in the depletion region between the P and N doped silicon. The electron and hole in the pair are separated by the electric field present in the depletion region, and generate a photo-current (or charge) which is amplified to measure the light levels. Any electron/hole pairs generated outside of the depletion region recombine, and do not contribute to the detected photocurrent. Short wavelengths in the UV range are absorbed strongly near the surface of the silicon before reaching the depletion region, and longer near IR wavelengths penetrate deeply into the silicon, and are often absorbed under the depletion region.

As a result, typical silicon detectors, lose sensitivity in the UV and near IR wavelengths. The sensitivity of a silicon imaging to near IR light, can be improved by manufacturing the detector with diodes that have deeper depletion regions, or by extending the depletion region using externally generated bias voltages. This increase in near IR sensitivity usually comes at the expense of some loss in sensitivity at the UV and blue ends of the spectrum.

3.8 Stacked Set Pixel Detectors

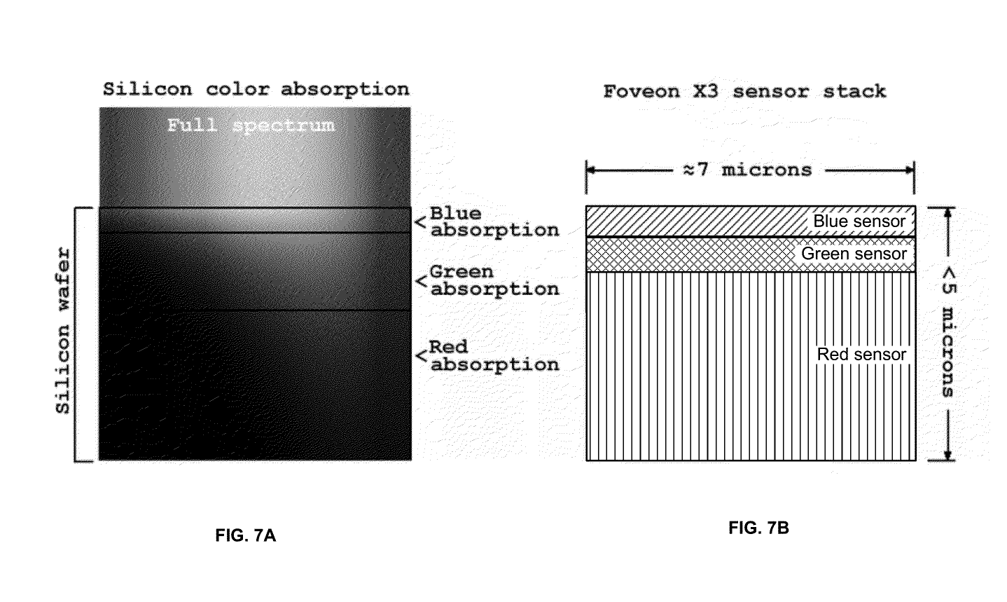

FIGS. 7A and 7B illustrate two different views of the approximate charge collection regions from a Foveon X3 stacked set pixel detector, according to one embodiment. Silicon stacked set pixel detectors rely on the fact that blue photons are absorbed near the surface of the silicon, green a little deeper, and red deeper still. By structuring electrodes to read out charge generated at different depths, color information can be generated from a single pixel.

The imaging system may use a modified stacked set pixel detector could be used in capturing iris images. The modification adds a fourth charge collector below the red detector to capture near IR information. An advantage of stacked set pixel detectors is that they are completely immune to color aliasing, and delivers true RGB estimates for each pixel without the need to incorporate information form adjacent pixels. This allows for finer granularity spatial resolution.

3.9. Dedicated Near IR Pixel Filter

A typical color detector uses a Bayer (or some variant) filter to allow different pixels or subpixels to detect different colors. Each color filter ensures that the underlying pixel sees only photons from a narrow range of wavelengths at the filter color. To generate color images from the readout of these chips, a convolution operation is performed which combines the image intensity from a number of adjacent pixels to estimate the image color over each pixel.

One embodiment of the imaging system uses a detector that includes a modified Bayer filter on top of the detector surface that includes IR filters for some pixels or subpixels. Changing the filter arrangement to an RGBI (red, green, blue, infrared) arrangement would allow simultaneous color and IR imaging. In one embodiment, the imaging system uses an Omnivision OV4682 detector beneath the modified (RGBI) Bayer filter.

One drawback with Bayer filters is that they work best for color areas which do not vary rapidly over the detector area in color or in brightness, so that adjacent pixels see the same color and brightness. If the image itself varies significantly at the pixel pitch of the detector, the color estimation algorithm will not be able to distinguish image brightness variation from color variation and incorrect colors can be estimated for the underlying image. This effect is known as color aliasing. This problem can be addressed by limiting the resolution of the lens, such that it cannot resolve picture elements as small as a pixel. Using this approach there is an inherent tradeoff between image resolution and color rendering accuracy.

To address this issue in iris imaging, in one embodiment, the imaging system uses the light signal received from all four channels (red, green, and blue in addition to IR) in order to maintain the highest possible spatial resolution. It is possible to receive signal through the RGB pixels or subpixel filters as these filters still transfer a significant amount of near IR light through, particularly at wavelengths such as 850 nm. As is discussed above and below, capture of images with and without near IR illumination and subtraction of those images can be used in conjunction with this capture of light through all four channels to provide a very high spatial resolution iris image.

Using this RGBI filter, in one embodiment, the RGBI filter replaces the notch IR filter introduced above. In another embodiment, the RGBI filter may be used in conjunction with the notch IR filter introduced above.

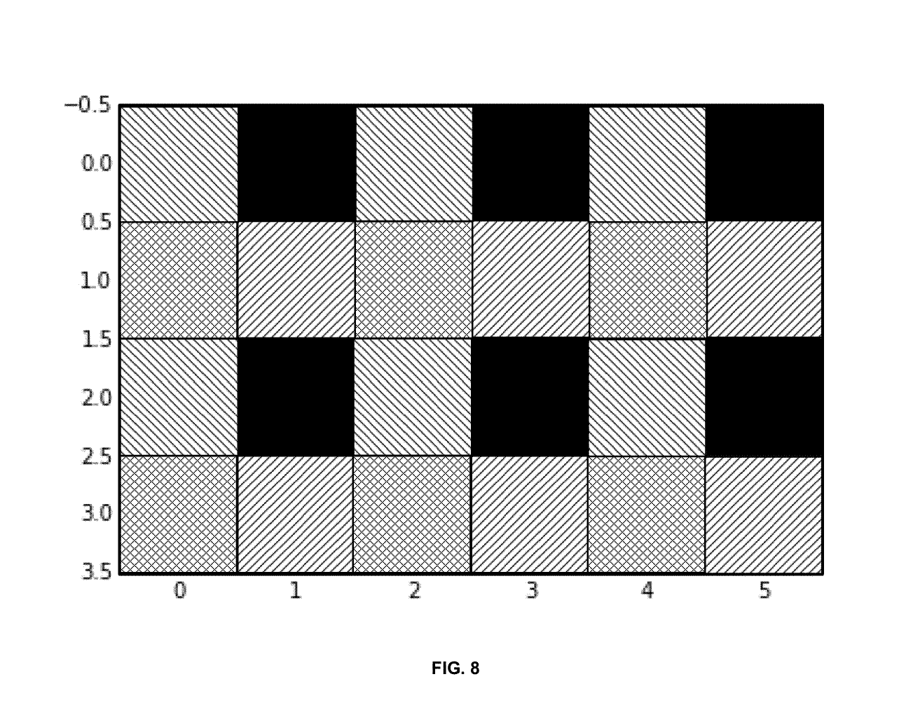

FIG. 8 illustrates an example modified Bayer filter to include color and near IR filters, according to one embodiment. The actual layout of the mask may vary by implementation.

### In [40]:

Bayer=[[[1.0,0,0],[0,1.0,0],[1.0,0,0],[0,1.0,0],[1.0,0,0],[0,1.0,0]], [[0,1.0,0],[0,0,1.0],[0,1.0,0],[0,0,1.0],[0,1.0,0],[0,0,1.0]], [[1.0,0,0],[0,1.0,0],[1.0,0,0],[0,1.0,0],[1.0,0,0],[0,1.0,0]], [[0,1.0,0],[0,0,1.0],[0,1.0,0],[0,0,1.0],[0,1.0,0],[0,0,1.0]]]

plt.imshow(Bayer,interpolation=‘nearest’)

In this example, half of the green filters have been replaced with near IR filters. In one embodiment, these near IR filters are notch filters as discussed above. There are many other possible arrangements of filters that could be used. In this example, a modified convolution filter would be used to generate the color information and the near IR image would be read directly from the near IR filter pixels. The optimal choice in this case would be to use a filter that blocks all visible light and just lets through near IR wavelengths. However alternative arrangements could work, even if the filter only partially blocked some visible wavelengths, a suitable convolution mask could still extract an estimate for IR intensity, but the signal would certainly be more noisy.

Alternatively, as introduced above some color filters admit IR light. The signal from the IR pixels could be used to subtract the IR signal contribution from the color filter pixels, thus restoring color balance and saturation even in the presence of IR illumination. In this situation an optimal estimator could essentially recover a four color intensity (RGBI) estimate for each pixel, the RGB component used to render a conventional color image and the I component used to render an IR image and provide IR intensity.

As another example:
In [44]: irb=[[[1.0,0,0],[0,0,0],[1.0,0,0],[0,0.0,0],[1.0,0,0],[0,0.0,0]], [[0,1.0,0],[0,0,1.0],[0,1.0,0],[0,0,1.0],[0,1.0,0],[0,0,1.0]], [[1.0,0,0],[0,0.0,0],[1.0,0,0],[0,0.0,0],[1.0,0,0],[0,0.0,0]], [[0,1.0,0],[0,0,1.0],[0,1.0,0],[0,0,1.0],[0,1.0,0],[0,0,1.0]]]

plt.imshow(irb,interpolation=‘nearest’)

3.10. WOI Controls

Most modern detectors allow for flexible on-chip binning to modify the effective resolution and window of interest (WOI) control which allows for only a subset of pixels to be read. These controls are typically used to allow for still-image and video camera functionality from the same detector, while also allowing for some level of digital zoom.

In one implementation, the imaging system may make use of WOI controls to optimize image capture for iris imaging. As an example, a typical detector may be able to read out pixels at a rate of the order of 3×10⁸ pixels per second, which allows for reading a VGA sized frame (640×480 pixels) in about 1 ms. The VGA frame size is the minimum size of an ISO standard-compliant iris image, but in practice, the frame size could be arbitrarily restricted to the order of 256×256 pixels, and still obtain an image which meets ISO quality specifications in all respects except for the size. This smaller frame would be readable in 200 μsec. Consequently, much higher than standard frame rates can be achieved by restricting the WOI. Captured images could then be upscaled to standard-size images after the fact.

Further, some detectors allow more than one simultaneous WOI to be defined, which would allow for iris images of both eyes of a human subject to be captured in the same exposure.

3.10.1. Iris Image Capture Process Using WOI Controls

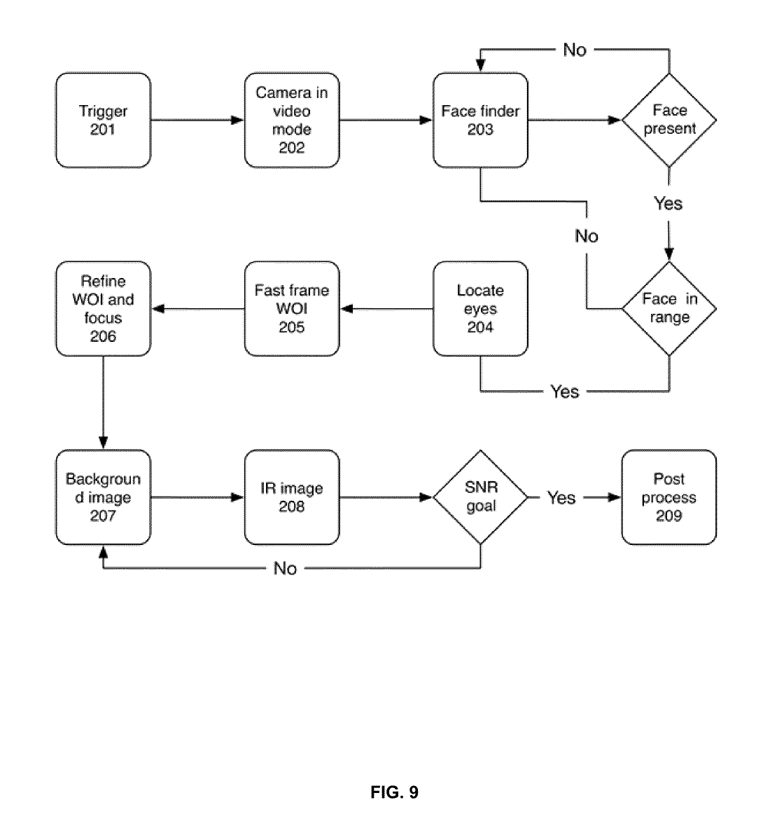

FIG. 9 illustrates a process for capturing iris images using WOI controls, according to one embodiment. Iris image capture is activated 201 by a trigger. The trigger will vary by implementation, an example includes: (1) A software or hardware button push, or other explicit action from the user; (2) A message from, or a side effect of the use of another application. For instance a banking application might request an authentication, which would display appropriate instructions on the screen and trigger the capture process; (3) Recognition of a login attempt to a web site or similar action may prompt iris recognition to enable a username and password to be retrieved from a secure key-ring that is unlocked by the iris biometric; (4) Activation via a Bluetooth, near field communication (NFC), wireless radio (WIFI), or cellular communication when a handheld device interacts with another device that requires authentication.

Automated systems such as automated teller machine (ATM) may activate 201 the iris image capture differently. In one embodiment, the imaging system looks for subject's faces continuously. In another embodiment, a hardware device such as a pressure mat or a range or proximity sensor may trigger activation 201. In another embodiment, a separate device may be used to trigger activation, such as insertion of an identification card into a card reader.

Once activation 201 has occurred, the imaging system places the detector in video mode 202 and begins capture of a video data stream. At the start of the capture process, the focus of the camera may be set to the midpoint of the expected capture volume. In one embodiment, on-chip signal binning is used to put the camera in full color VGA or 720P video mode. VGA mode would provide adequate resolution to find the eye locations to sufficient accuracy, but higher resolution video may also be usable. The detector is binned down to allow a higher frame rate and improve responsivity of the system. In one embodiment, the imaging system may activate a near IR illumination at a relatively low power, and detect the presence of a near IR glint from the subject's iris to assist in finding the eye location.

The mobile computing device runs a face finding algorithm 202 on the video data stream received from the imaging system. The face finding algorithm could be run in software on a general purpose CPU, or in a special purpose graphics processing array, or be implemented on a dedicated hardware processor. Face finding typically runs until a face was found, or until a timeout period has elapsed. If the camera and image processing capability has focusing capability, the camera focus could be adjusted concurrently while the face finding software is operating. If no face is found, the iris image capture process may exit.

If a face is found, the mobile computing device determines 203 whether the face is within range for iris imaging. This can be done in several ways. For example, the video data stream can analyze images of the subject's face to gauge the distance to the face from the mobile computing device. In one embodiment, the face distance can be determined by measuring the size of the face in the image, or by measuring some property of the face such as the inter-pupillary distance. For most of the adult population, the inter-pupillary distance is within a narrow range, and thus the distance as it appears on the face image can be used to extrapolate the distance to the face. As another example, if the focus position of the lens of the imaging system can be read out, then the focus position of the lens can measure the distance to the subject's face with quite good accuracy. For lenses that have a repeatable position control that drifts slowly over time and or temperature, the focus distance can be continuously re-calibrated by noting the focus position and size of the iris images over time and temperature.

If the face is not within range for iris image capture, feedback may be provided to the user through the face finding software operating on the mobile computing device to reposition the mobile computing device into an appropriate range.

If the face is within range for iris image capture, the face finding software reports 204 the location of one or both of the eyes within one or more images of the received video stream. The eye locations can be used to define one or two WOI for iris imaging, depending upon whether one or two eyes is within the captured face image and/or depending upon whether one iris image is to be captured at a time. Many currently available detectors do not have flexible WOI control, so some advantage may be obtained by redesigning the control circuitry to optimize WOI readout.

The detector 205 is switched to a fast framing WOI mode using the WOI previously defined 204. The imaging system then refines 206 the iris focus and WOI to identify a better focus for iris image capture. Even if active focus adjustment has been used during face finding, a much more accurate focus is used to capture iris images. In one embodiment, the imaging system uses the near IR glint reflected from eye cornea to refine the iris focus. In this embodiment, the imaging system turns on the near IR illuminator at a low intensity such that it produces a strong glint image, but not so strong so as to cause the glint to saturate the detector. The detector integration level may be reduced in order to cut down on background light and prevent saturation. The detector's integration time may be set to a value that represents the best tradeoff between image SNR and motion blur suppression.

In one embodiment, the refining 206 of the iris focus can be performed by stepping through different focus positions as described in co-pending U.S. patent application Ser. No. 13/783,838, the contents of which are incorporated by reference herein in their entirety.

Once the focus is determined, a background iris image is captured 207. To capture the background image I₁, the near IR illuminator is turned off and an image of the iris WOI is captured. Depending upon the implementation, the capture of the background image may also capture data outside the WOI, however this additional data is not required and may be an artifact of the exact capture process used.

The near IR image is also captured 208. To capture near IR image, the near IR illuminator is turned on to a high (e.g., full) brightness and a second image I₂ is taken with a same exposure and detector gain settings as is used for capturing the background image 207. Although this discussion describes the background image I₁ as being captured first and the near IR image I₂ as being captured second, this order is arbitrary and may be reversed in practice.

The background 207 and near IR 208 images are captured as close together in time as possible. Modern detectors are able to run at around 200 Mpixels per second, usually split into 50 Mpixels per second for each of 4 separate readout amplifiers, each of which is wired to one color (e.g., red, blue, and two separate green) output. If an iris image can be defined in an area of approximately 200 pixels square, then an effective frame time of 200 μsec or 1/500^{th} of a second can be achieved. Actual readout times would be a little slower, since in practice some line over scan (e.g., 50 pixels) is needed to set the dark value, and to stabilize the readout signal chain. At this frame rate it is possible to take a flash on and flash off image quickly enough to freeze motion and achieve a good image subtraction.

In one embodiment, the background 207 and iris 208 image capture steps are together repeated for more than one iteration (e.g., more than one pair of background and iris images are captured). In this implementation, the exposure time for the capture of each pair is reduced relative to an implementation where only one pair is captured, as discussed previously. The read noise of most CMOS detectors is small compared to the background photon noise, even for a 1 ms exposure, consequently, there is no significant penalty noise for taking the image using multiple short exposures. An advantage of using multiple exposures is that the images can be re-centered using the iris/illuminator glint as a reference before performing the subtraction between, therefore significantly reducing image motion blur. A disadvantage of taking multiple exposures is that off-the-shelf detectors may not be optimized for good performance in this mode of operation.

If multiple pairs of images are taken, successive near IR image captures can be re-centered within the WOI by identifying the location of the cornea glint in each near IR image. The position of the background images in each pair can be estimated using interpolation from the preceding and following near IR images based on the time between capture. Alternately, the positions of the background images can be determined if the near IR illuminator is turned on at low brightness (e.g., 1% of full power) during background image capture. This allows for the background image to be centered using the glint location, without significantly impacting the iris signal.

Post processing 209 is performed on the background I₁ and near IR I₂ images. If a single pair of images were captured, post-processing 209 subtracts the near IR image I₂ from the background image according to:

Iₛ=I₂−I₁

If multiple pairs of background and near IR images were captured, post processing 209 subtracts the near IR images from the background images according to:

$${I}_{s} = {I}_{2} - {I}_{1} + {I}_{2 N} - {I}_{2 N - 1} + {\sum }_{N = 2}^{n = N - 1} {I}_{2 n} + ( {I}_{2 n - 1} + {I}_{2 n + 1} ) / 2 \qquad ( 2 )$$

A major factor in estimating the SNR of the final subtracted iris image is the brightness of the background image. Consequently, the SNR can be estimated prior to creation of the subtracted iris image by reading the light level observed during face finding 203.

### 4. Example Imaging System

4.1. Example Detector Parameters

Basic parameters for a dual purpose (iris and color image) camera are set out in Table 2. These parameters are example only and could be varied in actual application. These parameters are for example only and could be varied depending on requirements and technical or cost constraints. Some of these parameters have been derived from the ISO19794-6 standard (herein referred to as the ISO standard) which sets forth minimum requirements for capturing valid iris images.

TABLE 2

| Parameter | Symbol | Value (unit) | | Notes |
|---|---|---|---|---|
| Minimum Pixel | lₛ | 70 | (μm) | Derived from ISO |
| Size on Iris | | | | standard |
| Pixel Density | | >140 | (pixels/cm) | Derived from ISO |
| | | | | standard |
| Pixel Pitch | | 14 | (pixels/mm) | Derived from ISO |
| on the Iris/ | | | | standard. The |
| Minimum | | | | average diameter of |
| Sampling | | | | the adult iris is |
| Resolution | | | | about 12 mm, so |
| | | | | this corresponds to |
| | | | | about 170 pixels |
| | | | | across an average |
| | | | | iris. |
| Nominal Max | l_{z} | 25-30 | (cm) | Preliminary estimate |
| standoff | | | | for a good user |
| | | | | experience with the |
| | | | | iris camera |
| Cornea Radius | | 7.5 | (mm) | |
| of Curvature | | | | |
| Long axis | α | 1 | (radian) | Example field of |
| field of | | | | view based on |
| view | | | | existing smart phone |
| | | | | front facing phone |
| | | | | cameras. May vary |
| | | | | slightly (e.g., 60 |
| | | | | degrees) |
| Long Axis | l_{w} | 4.8 | (mm) | Example size based |
| Width of | | | | on existing smart |
| Detector | | | | phone front facing |
| | | | | cameras. Assumes |
| | | | | the aspect ratio |
| | | | | immediately below |
| Long Axis/ | | 16/9 | | |
| Short Axis | | | | |
| Aspect Ratio | | | | |
| F-Ratio of | | 3 | | |
| Imaging | | | | |
| Lens | | | | |
| Distortion | | <2 (pixels | | |
| | | over iris | | |
| | | diameter) | | |
| Minimum | MTF | >60% at 2 | | Modulation Transfer |
| sharpness | | line pairs | | Function. Based on |
| | | per mm | | ISO ISO 19794-6 |
| Gray levels | | 255 (in | | ISO standard does |
| | | image) | | not tightly specify |
| | | 128 (over iris | | how the range of |
| | | structure) | | each level is |
| | | | | defined. |
| Noise level | SNR | 20 | | Standard does not |
| | | | | quantify this |
| Operable | | 0-100,000 | (Lux) | Typically operation |
| Ambient Light | | | | occurs under 100- |
| Environment | | | | 1000 lux |
| Speed of | | <1 | (sec) | |
| Capture | | | | |

In one specific embodiment, the parameters from table 2 above allow for determination of the characteristics needed from a CMOS detector in order to capture valid iris images. For example, given the long axis field of view and the pixel size the total number of pixels in the detector nₚᵢₓ can be computed according to:

$${n}_{pix} = {( \frac{2 {l}_{z} tan α / 2}{{l}_{s}} )}^{2} \frac{9}{16} \qquad ( 3 )$$

which according to the example parameters of table 2 is 6.6 million pixels (Mpixels). The pixel size lₚᵢₓ can be computed from the number pixels according to:

lₚᵢₓ=sqrt(l_{w}²×9/(16nₚᵢₓ) (4a)

or according to

$${l}_{pix} = 70 μ {m ( \frac{w}{{l}_{z}} )}^{2} \qquad ( 4 b )$$

which gives a pixel size lₚᵢₓ of 1.3-1.4 microns (μm). The lens focal length l_{f} can be computed according to:

l_{f}=lₜ*lₚᵢₓ/lₛ (5)

which gives a focal length l_{f} of 5 mm.

Various implementations may use different parameters from those listed above that still meet the minimum requirements set forth by the ISO standard. Using different parameters creates tradeoffs in design performance. For instance, larger format detectors offer larger physical pixels resulting in more resolution for the iris image, but in turn use longer focal length lenses which are more difficult to package in the confined space provided by a mobile device.

4.2. SNR Calculation for Iris Imaging in Bright Sunlight

The most difficult situation for the iris imaging system is outside imaging, because the sun's illumination has a significant near IR component in addition to producing a strong visible signal. The near IR component interferes with white balance correction for portrait imaging. The visible component adds significant noise to iris images.

A practical worst case is where the iris is diffusely illuminated by reflected light from a high albedo environment, for instance whitewashed walls with an albedo of approximately 0.7, and an iris having a wavelength-independent albedo of 0.15. If the imaging system is able to capture an iris image with sufficient SNR under these conditions, it can be assumed it will also be able to function under less onerous conditions.

In one embodiment, the imaging system captures two iris images: (1) a first image under illumination by ambient light, then (2) a second image under illumination by ambient light and by an IR illuminator. The two images are then subtracted to generate the iris image. The images are taken close in time to avoid changes in the underlying image.

The main degradation in image quality is due to the noise introduced by the subtraction process. The expression for the per pixel SNR is given below:

$$SNR = \frac{ST}{\sqrt{( 2 BT + ST + 2 {R}^{2} )}} \qquad ( 6 )$$

where T is exposure time, S is signal level expressed in detected photoelectrons per second, B is background intensity expressed as detected photoelectrons per second, and R is read noise expressed in photoelectrons.

To calculate the signal level S, it is assumed that the near IR illuminator achieves an illumination level of 10 mW per square cm on the iris. This is a relatively low light level that can easily be achieved using an eye safe illuminator configuration. The power per unit area per unit time of IR iris illumination is a design parameter that can be adjusted. The signal level S can be computed according to:

$$N = \frac{{Al}_{p}^{2} π {r}_{l}^{2}}{2 π {l}_{z}^{2}} \int {n}_{LED} ( λ ) Q ( λ ) {f}_{c} ( λ ) {f}_{ir} ( λ ) d λ \qquad ( 7 )$$

where all of the parameters are the same as equation (1) above except n_{LED}(λ) is the near IR illuminator's spectrum (assuming an LED illuminator) expressed as number of photons per unit wavelength per second. The computed throughputs of the Bayer are shown in Table 3, assuming an albedo of 0.1.

TABLE 3

| | Red | Green | Blue | |
|---|---|---|---|---|
| Description | filter | filter | filter | Comment |
| Background | 553 | 584 | 257 | Signal from diffuse reflection of |
| | | | | sunlight illuminating iris |
| Corneal glint | 113 | 117 | 51 | Signal from diffuse reflection of |
| | | | | sunlight reflecting of cornea |
| Signal | 209 | 176 | 186 | Signal from IR illuminator |
| Noise | 41.1 | 41.3 | 30.5 | Total noise in subtracted image |
| SNR | 5.1 | 4.3 | 6.1 | SNR of resulting subtraction image |
| | | | | for a 1 ms exposure |

Table 3 illustrates reflected signal levels due to various sources, including diffuse reflection of sunlight from the illuminated iris (background), diffuse reflection of sunlight from the cornea, signal from the IR illuminator, noise in the subtracted image, and the SNR of a subtracted image assuming a 1 ms exposure.

These numbers illustrate that the cornea has a reflectivity of approximately 3%. The cornea acts as a mirror, reflecting an image of the scene that is observed by the subject. The cornea reflection therefore adds an additional signal that would be ⅕ of the iris signal in the worst-case situation. The subtraction process removes the cornea image, but the existence of the cornea image adds additional noise to the final image

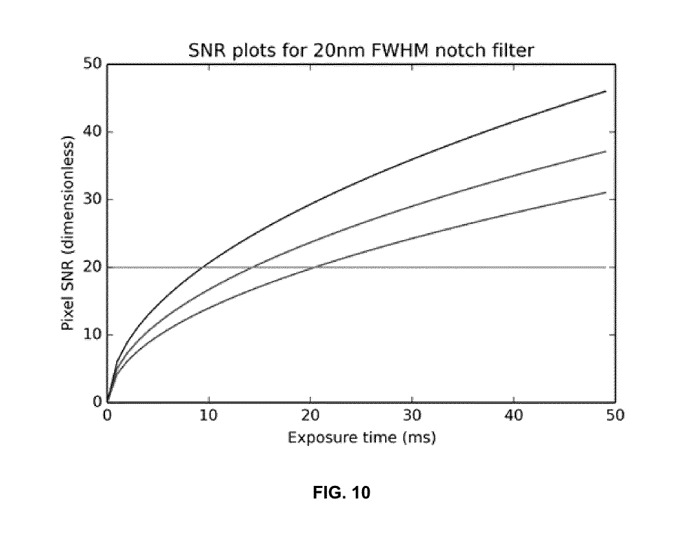

FIG. 10 plots the SNR for red, green, and blue Bayer filters as a function of exposure time, according to one embodiment. In this example, an exposure time of approximately 20 milliseconds (ms) gives an SNR of 20. Under most illumination circumstances a significantly shorter exposure time will give sufficient SNR. The length of exposure can be calculated by measuring the ambient light level. A subtracted iris image may be built from a single 20 ms background exposure and a 20 ms near IR illuminated exposure, or by taking a sequence of shorter exposure images alternately background and near IR illuminated and subtracting each pair of images.

4.3. Example SNR Calculation for Imaging System

The SNR of the imaging system can be characterized under various lighting conditions. In one example calculation, to model the SNR of the imaging system 120, the ground level solar spectral illumination can be modeled by a scaled black body spectral distribution, where the spectral density per Hz I can be calculated according to:

$$I ( v , T ) = \frac{2 {hv}^{3}}{{c}^{2}} {( {e}^{\frac{hv}{kT}} - 1 )}^{- 1} {WSr}^{- 1} {Hz}^{- 1} {m}^{- 2} \qquad ( 8 )$$

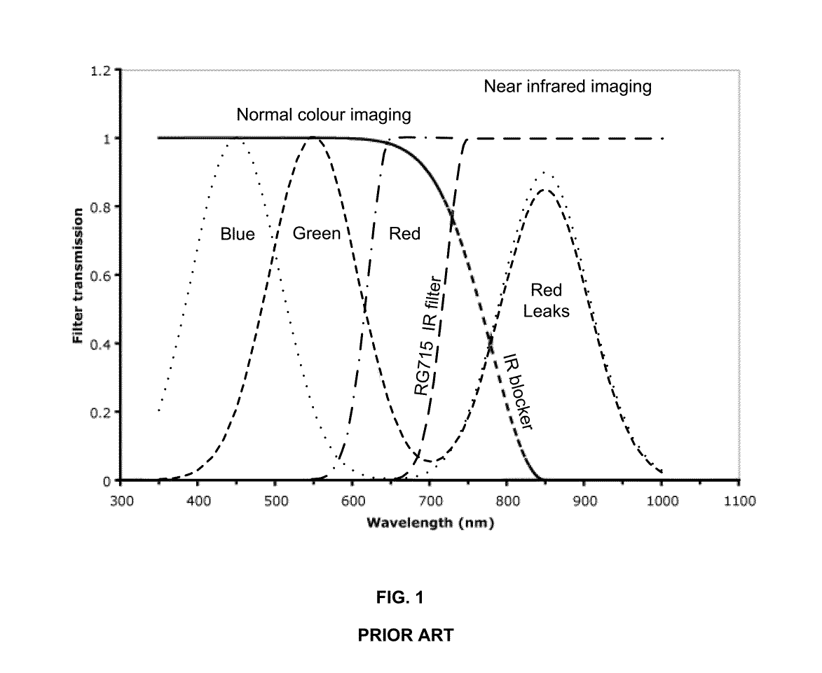

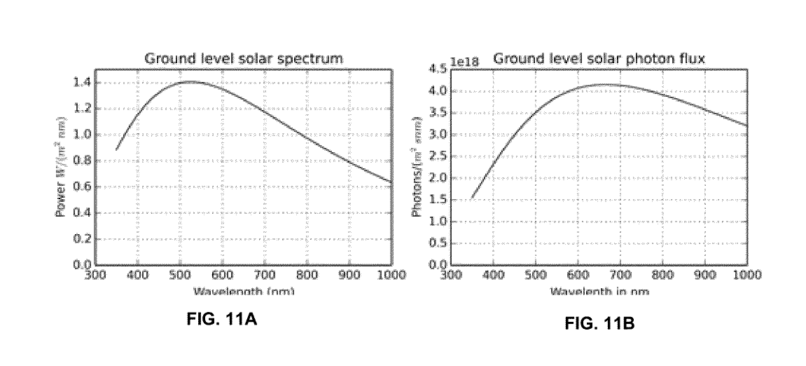

FIGS. 11A and 1B illustrates example scaled black body spectral distribution plotting Power (Watts per unit volume) and Photons (Count of photon flux per unit volume) as a function of wavelength for use in an SNR calculation of the imaging system, according to one embodiment. These distributions may be used to determine the parameters of the iris imaging system that allow for capture in daylight.

Define filter expressions which define the brightness

In [201]: def sbbs(v):
# Solar spectrum at frequency v.
# Surface temperature of the sun (actual surface temp is 5780, but
# This lower temperature gives the best black body fit
T=5780
k=1.38e−23
h=6.626e−34
c=3.0e8
solarRad=69600
earthOrbit=152e6
geometricExtinction=(solarRad/earthOrbit)**2
atmosLoss=0.6
return(atmosLoss*geometricExtinction*(2*h*(v**3)/(c**2))/(exp((h*v)/(k*T))−1))

def bfilt(lamb):
#Blue filter throughput estimate,
# lambda is wavelength in nm
return (exp(−((lamb−440e−9)**2/(2*50e−9**2)))+0.9*exp(−((lamb−850e−9)**2/(2*50e−9*

def gfilt(lamb):
#Green filter throughput estimate,
# lambda is wavelength in nm
return (exp(−((lamb−550e−9)**2/(2*50e−9**2)))+0.85*exp(−((lamb−850e−9)**2/(2*50e−9**

def rfilt(lamb):
#Red filter throuhgput estimate,
# lambda is wavelength in nm
red=exp(−((lamb−650e−9)**2/(2*50e−9**2)))
red=(lamb<650e−9)*red+(lamb>=650e−9)*1.0
return (red)

def irfilt(lamb):
ir=1−exp(−((lamb−650e−9)**2/(2*30e−9**2)))
ir=(lamb<650e−9)*0+(lamb>=650e−9)*ir
return (ir)

def irnotch(lamb,std):
# A IR blocking filter with a notch around 850 nm
center=850e−9
#notch=1−exp(−((lamb−650e−9)**2/(2*50e−9**2)))−exp(−((lamb−center)**2/(2*std**2)))
#notch=(lamb<650e−9)*0+(lamb>=650e−9)*notch
notch=irfilt(lamb)−exp(−((lamb−center)**2/(2*std**2)))
return (notch)

def siresp(lamb):
# Rough estimate of Si detector response
peakQE=0.6
sir=peakQE*exp(−((lamb−600e−9)**2/(2*120e−9**2)))
sir2=peakQE*exp(−((lamb−600e−9)**2/(2*180e−9**2)))
sir=(lamb<600e−9)*sir+(lamb>=600e−9)*sir2
return(sir)

Having defined the filters, look at the rough signal levels expected for the different filters responding to the solar spectrum. This is only an approximate number because the QE is measured at the photon level not at the power level. This will be fixed when the actual SNR is calculated.

In [218]: print(‘Total solar flux’,sum(I),‘W/m̂2’)
print(‘Total flux seen by blue filter and imager with ir filter’,
str.format(‘ {0:.1f}’,sum(I*bfilt(lam)*siresp(lam)*(1−irfilt(lam)))),‘W/m̂2’)
print(‘Total flux seen by blue filter and imager with ir notch filter’,
str.format(‘{0:.1 f}’,sum(I*bfilt(lam)*siresp(lam)*(1−irnotch(lam,notch)))),‘W/m̂2’)
print(‘Total flux seen by blue filter and imager with no ir filter’,
str.format(‘{0:.1f}’,sum(I*bfilt(lam)*siresp(lam))),‘W/m̂2’)
print(‘Total flux seen by green filter and imager with ir filter’,
str.format(‘{0:.1f}’,sum(I*gfilt(lam)*siresp(lam)*(1−irfilt(lam)))),‘W/m̂2’)
print(‘Total flux seen by green filter and imager with ir notch filter’,
str.format(‘{0:.1 f}’,sum(I*gfilt(lam)*siresp(lam)* (1−irnotch(lam,notch)))),‘W/m̂2’)
print(‘Total flux seen by green filter and imager with no ir filter’,
str.format(‘{0:.1f}’,sum(I*gfilt(lam)*siresp(lam))),‘W/m̂2’)
print(‘Total flux seen by red filter and imager with ir filter’,
str.format(‘{0:.1f}’,sum(I*rfilt(lam)*siresp(lam)*(1−irfilt(lam)))),‘W/m̂2’)
print(‘Total flux seen by red filter and imager with ir notch filter’,
str.format(‘{0:.1 f}’,sum(I*rfilt(lam)*siresp(lam)*(1−irnotch(lam,notch)))),‘W/m̂2’)
print(‘Total flux seen by red filter and imager with no ir filter’,
str.format(‘{0:.1 f}’,sum(I*rfilt(lam)*siresp(lam))),‘W/m̂2’)
(‘Total solar flux’, 1037.6398386746973, ‘W/m̂2’)
(‘Total flux seen by blue filter and imager with ir filter’, ‘48.1’, ‘W/m̂2’)
(‘Total flux seen by blue filter and imager with ir notch filter’, ‘56.3’, ‘W/m̂2’)
(‘Total flux seen by blue filter and imager with no ir filter’, ‘91.7’, ‘W/m̂2’)
(‘Total flux seen by green filter and imager with ir filter’, ‘114.1’, ‘W/m̂2’)
(‘Total flux seen by green filter and imager with ir notch filter’, ‘121.9’, ‘W/m̂2’)
(‘Total flux seen by green filter and imager with no ir filter’, ‘156.0’, ‘W/m̂2’)
(‘Total flux seen by red filter and imager with ir filter’, ‘106.0’, ‘W/m̂2’)
(‘Total flux seen by red filter and imager with ir notch filter’, ‘115.3’, ‘W/m̂2’)
(‘Total flux seen by red filter and imager with no ir filter’, ‘244.8’, ‘W/m̂2’)

For the iris specular reflection, the specular reflection of the sun is too bright to suppress and is thus ignored. It is assumed that the specular reflections of interest come from diffusely reflective white structure illuminated by the full sun. The corneal acts like a negative lens of focal length about 3.75 mm, which makes the objects pretty much at the focal length of the lens behind the cornea. The surface brightness of the objects is independent of their distance, but their size scales. Since the objects are diffuse, the same formula can be used for the diffuse object, except that the brightness is suppressed by the reflectivity of the cornea, which is about 3%.

### In [41]:

#Specular Reflectivity of the cornea
cornea_r=0.03
#Albedo of white surfaces, nothing reflects 100%
white_r=0.7
#Albedo of and iris in the visible (assume blue since that is worst case)
iris_r=0.15
#IR Albedo of iris
iris_ir_r=0.2
#Angular of pixel on far field, amount to surface seen by a single pixel
#Pixel is defined as 14 pixels per mm in this calculation
radiance=1/(pp*1000)**2
# How much of the light from the object reaches the camera
aperture=fl/lens_f_ratio
# Assuming that the light from the environment scatters into 2Pi steradians
# diffuser to lens throughput
lens_diffuse=0.125*(aperture/irr)**2
flux_b_ir=sum(photons*bfilt(lam)*siresp(lam)*(1−irfilt(lam)))*radiance*white_r*iris_r*lens_diffuse/1000
flux_b_irn=sum(photons*bfilt(lam)*siresp(lam)*(1−irnotch(lam,notch)))*radiance*white_r*iris_r*lens_diffuse/1000
flux_b_open=sum(photons*bfilt(lam)*siresp(lam))*radiance*white_r*iris_r*lens_diffuse/1000
flux_g_ir=sum(photons*gfilt(lam)*siresp(lam)*(1−irfilt(lam)))*radiance*white_r*iris_r*lens_diffuse/1000
flux_g_irn=sum(photons*gfilt(lam)*siresp(lam)*(1−irnotch(lam,notch)))*radiance*white_r*iris_r*lens_diffuse/1000
flux_g_open=sum(photons*gfilt(lam)*siresp(lam))*radiance*white_r*iris_r*lens diffuse/1000
flux_r_ir=sum(photons*rfilt(lam)*siresp(lam)*(1−irfilt(lam)))*radiance*white_r*iris_r*lens_diffuse/1000
flux_r_irn=sum(photons*rfilt(lam)*siresp(lam)*(1−irnotch(lam,notch)))*radiance*white_r*iris_r*lens_diffuse/1000
flux_r_open=sum(photons*rfilt(lam)*siresp(lam))*radiance*white_r*iris_r*lens_diffuse/1000
flux_ir_only=sum(photons*(1−irnotch_only(lam,notch))*siresp(lam))*radiance*iris_r*lens_diffuse/1000
flux_ir_only_r=sum(photons*(1−irnotch_only(lam,notch))*rfilt(lam)*siresp(lam))*radiance*iris_r*lens_diffuse/1000
flux_ir_only_g=sum(photons*(1−irnotch_only(lam,notch))*gfilt(lam)*siresp(lam))*radiance*iris_r*lens_diffuse/1000
flux_ir_only_b=sum(photons*(1−irnotch_only(lam,notch))*bfilt(lam)*siresp(lam))*radiance*iris_r*lens_diffuse/1000
print(‘iris signal Blue flux ir filter, notch ir, no ir filter’,
   ‘ {0:.0f} {1:.0f}
{2:.0f}’.format(flux_b_ir,flux_b_irn,flux_b_open),‘photons/pixel/ms’)
print(‘iris signal Green flux ir filter, notch ir, no ir filter’,
   ‘ {0:.0f} {1:.0f}
{2:.0f}’.format(flux_g_ir,flux_g_irn,flux_g_open),‘photons/pixel/ms’)
print(‘iris signal Red flux ir filter, notch ir, no ir filter’,
   ‘ {0:.0f} {1:.0f}
{2:.0f}’.format(flux_r_ir,flux_r_irn,flux_r_open),‘photons/pixel/ms’)
print(‘ir only filter filter, notch ir, visible blocker
{0:.0f}’.format(flux_ir_only),‘photons/pixel/ms’)
print(‘ir only filter filter, red, green, blue filter {0:.0f} {1:.0f}
{2:.0f}’.format(flux_ir_only_r,flux_ir_only_g,flux_ir_only_b),‘photons/pixel/ms’)
# Corneal reflection calculation
# Light from a single pixel on the cornea (dimensions pp*pp) expands at an angle
that is set by half of the radius of
# curvature of the cornea diffusing object is at infinity. Some of this light cone is
intersected by the lens aperture.
spec_b_ir=flux_b_ir*cornea_r/iris_r
spec_b_irn=flux_b_irn*cornea_r/iris_r
spec_b_open=flux_b_open*cornea_r/iris_r
spec_g_ir=flux_g_ir*cornea_r/iris_r
spec_g_irn=flux_g_irn*cornea_r/iris_r
spec_g_open=flux_g_open*cornea_r/iris_r
spec_r_ir=flux_r_ir*cornea_r/iris_r
spec_r_irn=flux_r_irn*cornea_r/iris_r
spec_r_open=flux_r_open*cornea_r/iris_r
spec_ir_only=flux_ir_only*cornea_r/iris_r
print(‘Cornea signal Blue flux ir filter, notch ir, no ir filter’,
   ‘{0:.0f} {1:.0f}
{2:.0f}’.format(spec_b_ir,spec_b_irn,spec_b_open),‘photons/pixel/ms’)
print(‘Cornea signal Blue flux ir filter, notch ir, no ir filter’,
   ‘{0:.0f} {1:.0f}
{2:.0f}’.format(spec_g_ir,spec_g_irn,spec_g_open),‘photons/pixel/ms’)
print(‘Cornea signal Blue flux ir filter, notch ir, no ir filter’,
   ‘{0:.0f} {1:.0f}
{2:.0f}’.format(spec_r_ir,spec_r_irn,spec_r_open),‘photons/pixel/ms’)
print(‘Cornea ir only filter filter, notch ir, visible blocker
{0:.0f}’.format(spec_ir_only),‘photons/pixel/ms’)
(‘iris signal Blue flux ir filter, notch ir, no ir filter’, ‘254 329 662’, ‘photons/pixel/ms’) (‘iris signal Green flux ir filter, notch ir, no ir filter’, ‘717 788 1106’, ‘photons/pixel/ms’) (‘iris signal Red flux ir filter, notch ir, no ir filter’, ‘754 839 1977’, ‘photons/pixel/ms’) (‘ir only filter filter, notch ir, visible blocker 122’, ‘photons/pixel/ms’) (‘ir only filter filter, red, green, blue filter 122 101 107’, ‘photons/pixel/ms’) (‘Cornea signal Blue flux ir filter, notch ir, no ir filter’, ‘51 66 132’, ‘photons/pixel/ms’) (‘Cornea signal Blue flux ir filter, notch ir, no ir filter’, ‘143 158 221’, ‘photons/pixel/ms’) (‘Cornea signal Blue flux ir filter, notch ir, no ir filter’, ‘151 168 395’, ‘photons/pixel/ms’) (‘ir only filter filter, notch ir, visible blocker 24’, ‘photons/pixel/ms’)

For the purposes of argument assume a single near IR LED illuminator with a collimating lens, and the detected photons/ms per pixel are computed.

### In [49]:

# Calculate the expected photon flux from a single LED
# LED total radiated power in W
led_power=0 0.55
# LED lens divergence in radians
led_fwhm=0.33
# LED lens efficiency, how much of the light from the LED is captured by the collimator lens
led_efficiency=0.5
# Calculate the power on the subject in W/cm2
iris_power_density=led_power/(3.14/4*(led_fwhm*irr/10)**2)
pixel_power=(iris_power_density/(10*pp)**2)*iris_ir_r*lens_diffuse
#Normalize the model diode spectrum to reflect the power on a single pixel
diode_spec=pixel_power*sdiode(lam)/sum(sdiode(lam))
led_pixel_phot=diode_spec*siresp(lam)*lam/(6.626e−34*3e8)
led_pixel_phot_notch=diode_spec*siresp(lam)*(1−irnotch(lam,notch))*lam/(6.626e−34*3e8)
print(‘Iris power density {0:.6f} $$W/cm̂2$$’.format(iris_power_density))
print(‘Pixel power density on detector {0:.6f}
   $$pW$$’.format(pixel_power*10e12))
print(‘Pixel detected photons (no filter) {0:.1 f}
phot/pixel/ms’.format(sum(led_pixel_phot)/1000))
print(‘Pixel detected photons (notch filter) {0:0.1f}
phot/pixel/ms’.format(sum(led_pixel_phot_notch)/1000))
#plot(lam*1e9,led_pixel_phot)
Iris power density 0.010294 $$W/cm̂2$$
Pixel power density on detector 4.376710 $$pW$$ Pixel detected photons (no filter) 440.2 phot/pixel/ms Pixel detected photons (notch filter) 225.2 phot/pixel/ms

### In [17]:

1e−6/(6.626e−34*3e8)

### Out[17]:

5.03068719187041e+18>

The following is an example SNR calculation where two short exposure images are subtracted to derive a iris image. Use the iris image SNR, compared to the SNR that would be obtained with a dedicated IR camera. Use the red bias signal that is added to the color image and estimate if this is fixable by post-processing. Assume the following parameters defined below. Also assume a worst case scenario where maximum glint illumination is present that is to be removed to obtain the iris image

### In [80]:

# Assume that the notch filter has h std deviation given by notch
print(‘Standard deviation of IR notch filter is {0:.1f} nm’.format(notch*1.0e9))
# Suppress visible spectrum to this proportion of original brightness visible_suppress=1.0
print(‘Visible spectrum is reduced by this factor {0:.1f}’.format(visible_suppress)) exposure_time=1
print(‘Exposure time for images is {0:.1f}ms’.format(exposure_time))
detector_read_noise=8
print(‘Assume detector read noise is {0:.1f} e−’.format(detector_read_noise));
# Compute the signal with No IR illumination (rgb)
background=array([flux_r_irn+spec_r_irn,flux_g_irn+spec_g_irn,flux_b_irn+spec_b_irn])
signal=array([sum(led_pixel_phot_notch*rfilt(lam)/1000),sum(led_pixel_phot_not
ch*gfilt(lam)/1000),sum(led_pixel_phot_notch*bfilt(lam)/1000)])
noise=sqrt(2*background+signal+2*detector read noise**2)
noise_dedicated=sqrt(signal+detector read noise**2)
snr=signal/noise
print(‘Background’,background)
print(‘Signal’,signal)
print(‘Noise’,noise)
print(‘SNR’,snr)
print(‘Dedicated iris camera SNR’, signal/noise_dedicated)

Standard deviation of IR notch filter is 10.0 nm. Visible spectrum is reduced by this factor 1.0. Exposure time for images is 1.0 ms. All numbers listed for individual filters in the order red, green, blue. Assume detector read noise is 8.0 e−(‘Background’, array([1006.88873168, 945.53390612, 395.38317034])) (‘Signal’, array([225.17732412, 188.33649032, 199.4046027])) (‘Noise’, array([48.65135956, 46.98302143, 33.43906314])) (‘SNR’, array([4.62838708, 4.00860746, 5.96322337])) (‘Dedicated iris camera SNR’, array([13.24166317, 11.85617071, 12.28636743])).

### 5. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe certain operations, such as the subtraction of background exposures from iris exposures from each other to generate iris images, in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof. In one embodiment, a software module for carrying out the described operations is implemented with a computer program product comprising a non-transitory computer-readable medium containing computer program code, which can be executed by a computer processor for performing any or all of the steps, operations, or processes described.

Embodiments of the invention may also relate to a mobile computing device for performing the operations herein. This device may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process (e.g., an iris image), where the information is stored on a non-transitory, tangible computer readable storage medium and may include any embodiment of a computer program product or other data combination described herein.

1. An iris imaging system comprising:
a near infrared (IR) illuminator for illuminating a subject's iris with near infrared light comprising an 850 nanometer (nm) wavelength;
a detector for receiving visible and near IR light reflected from the iris;
a notch IR filter positioned along the optical path between the detector and the iris, the notch IR filter blocking a majority of light except for wavelengths near a transmission notch centered within 20 nm of the 850 nm wavelength and having a full width half maximum (FWHM) of less than or equal to 20 nm, the transmission notch transmitting a majority of light within the FWHM; and
a mobile computing device comprising a processor and a non-transitory computer readable storage medium, the medium storing computer program instructions configured to cause the iris imaging system to capture an iris image, the instructions causing the iris imaging system to:
capture a background exposure of the subject while the near IR illuminator is either deactivated or activated at less than 30% of full power;
capture a near IR exposure of the subject while the near IR illuminator is activated; and
subtract the background exposure from the near IR exposure to generate the iris image of the iris.

2. The iris imaging system of claim 1, wherein the near IR illuminator is a light emitting diode (LED).

3. The iris imaging system of claim 1, wherein the FWHM is less than or equal to 10 nm.

4. The iris imaging system of claim 3, wherein the near IR illuminator comprises at least one laser.

5. The iris imaging system of claim 1, wherein the instructions further cause the iris imaging system to:
capture a plurality of exposure pairs, each exposure pair comprising one of a plurality of background exposures, and one of a plurality of near IR exposures;
subtract the background exposure from the near IR exposure of each pair to generate a portion of the iris image based on the subtraction of each pair.

6. The iris imaging system of claim 5, wherein the instructions further cause the iris imaging system to:
track a physical motion of the iris imaging system based on infrared light received in the near IR exposure of each pair;
align the background exposure with the near IR exposure of each pair based on the tracked physical motion.

7. The iris imaging system of claim 6, wherein tracking the physical motion comprises interpolating the tracked physical motion based on infrared light received in the near IR exposure of each pair and a subsequent or a previous infrared light received in a subsequent or previous near IR exposure.

8. The iris imaging system of claim 1, wherein the iris image is captured at a standoff distance of between 25-30 centimeters.

9. The iris imaging system of claim 1, wherein the detector comprises a global shutter detector wherein all pixels of the detector begin and end integration at a same time.

10. The iris imaging system of claim 1, wherein the detector comprises a comparator electrically coupled to each pixel of the detector, the comparator flipping whenever a threshold number of pixels have been received, and wherein each comparator is associated with a counter that counts a number of comparator flips.

11. The iris imaging system of claim 1, wherein the detector comprises electrical circuitry configured to read each pixel after reset, after a first integration time while the near IR illuminator is not activated, and after a second integration time while the near IR illuminator is activated.

12. The iris imaging system of claim 1, wherein the iris imaging system comprises a dichroic beam splitter splitting visible incident light and IR incident light onto separate optical paths, and the detector comprises a visible light detector chip receiving the visible incident light as well as a IR light detector chip receiving the IR incident light.

13. The iris imaging system of claim 1, wherein the detector comprises a stacked set pixel detector comprising a blue sensor near an outer surface of the detector facing the iris, a green sensor beneath the blue sensor, a red sensor beneath the green sensor, and an IR sensor beneath the red sensor.

14. The iris imaging system of claim 1, wherein the iris imaging system comprises a modified Bayer filter between a surface of the detector and the iris, the modified Bayer filter comprising a plurality green filters for a first subset of pixels of the detector, a plurality of red filters for a second subset of the pixels, a plurality of blue filters for a third subset of the pixels, and a plurality of IR filters for a fourth subset of the pixels.

15. The iris imaging system of claim 1, wherein the background exposure and the near IR exposure comprise data regarding a subset of all pixels of the detector within a window of interest (WOI).

16. The iris imaging system of claim 1, wherein the WOI comprises a 256×256 block of pixels of the detector.

17. The iris imaging system of claim 1, wherein the WOI comprises a 640×480 block of pixels of the detector.

18. The iris imaging system of claim 1, wherein the notch IR filter comprises a plurality of transmission notches, a first of the transmission notches being the transmission notch centered within 20 nm of the 850 nm wavelength, a second of the transmission notches centered at a 780 nm wavelength of light.

19. An iris imaging system comprising:
a plurality of illuminators for illuminating a subject's iris with light, a first of the illuminators centered at a 850 nanometer (nm) wavelength, a second of the illuminators centered at a 750 nm wavelength;
a detector for receiving visible and near IR light reflected from the iris;
a notch IR filter comprising a plurality of transmission notches, the notch IR filter positioned along the optical path between the detector and the iris, the notch IR filter blocking a majority of light except for wavelengths near any of the transmission notches, a first of the transmission notches centered within 20 nm of the 850 nm wavelength, a second of the transmission notches centered within 20 nm of the 780 nm wavelength; and
a mobile computing device comprising a processor and a non-transitory computer readable storage medium, the medium storing computer program instructions configured to cause the iris imaging system to capture an iris image, the instructions causing the iris imaging system to:
capture a background exposure of the subject while the near IR illuminators are deactivated;
capture a near IR exposure of the subject while the near IR illuminators are activated; and
subtract the background exposure from the near IR exposure to generate the iris image of the iris.

20. The iris imaging system of claim 19, wherein one of the near IR illuminators is a light emitting diode (LED) illuminator, and another of the illuminators is a laser illuminator.
